(19) United States
(12) Patent Application Publication
(10) Pub. No.: US 2026/0360561 A1
(43) Pub. Date: Nov. 12, 2026

(54) SYSTEM AND METHOD FOR EXTRACTING AND VALIDATING CLINICAL RELATIONSHIP TRIPLETS FROM BIOMEDICAL LITERATURE

(71) Applicant: HEALTHY ASIAN KITCHEN LIMITED, London (GB)

(72) Inventors: Abishek Rao, Udupi (IN); Aadhar Chhabra, London (GB)

(73) Assignee: HEALTHY ASIAN KITCHEN LIMITED, London (GB)

(21) Appl. No.: 19/203,309

(22) Filed: May 9, 2025

(51) Int. Cl. G16H 50/70; G06F 40/169; G06F 40/295; G06F 40/30; G06F 40/40; G16H 20/60

(52) U.S. Cl. CPC G16H 50/70; G06F 40/169; G06F 40/295; G06F 40/30; G06F 40/40; G16H 20/60

(57) ABSTRACT

The present disclosure provides a system and a computer-implemented method for extracting and validating clinical relationship triplets from a biomedical literature. The method includes retrieving, by a processor associated with a system, biomedical abstracts from one or more databases associated with the biomedical literature using one or more automated tools. The method includes extracting, by the processor, relationship triplets from the retrieved biomedical abstracts. Further, the method includes validating, by the processor, the extracted relationship triplets through a multi-stage evaluation framework, and facilitating, by the processor, personalized dietary recommendations to one or more users by structuring and outputting the validated relationship triplets.

### TECHNICAL FIELD

The embodiments of the present disclosure generally relate to a field of natural language processing (NLP), machine learning, and biomedical text mining. More specifically, the present disclosure pertains to a system and a method for extracting, analysing, and validating clinical relationship triplets from biomedical literature using a combination of transformer-based models, Large Language Models (LLMs), machine learning classifiers, and multi-stage validation techniques.

### BACKGROUND

Biomedical relationship extraction has evolved from traditional rule-based approaches to modern machine-learning techniques. Traditional methods relied on rule-based systems and classical machine learning algorithms, such as Conditional Random Fields (CRFs) and Support Vector Machines (SVMs), which required manual feature engineering and extensive annotated datasets. These techniques are labour-intensive and lack adaptability across different biomedical subdomains.

In contrast, modern approaches leverage transformer-based models, which automatically learn contextual representations from vast biomedical texts. These models have significantly improved Named Entity Recognition (NER) and relationship extraction by capturing complex linguistic patterns. Additionally, Large Language Models (LLMs), particularly in zero-shot and few-shot settings, have introduced prompt engineering as a way to reduce dependence on large, domain-specific training datasets and simplify the extraction process.

Despite these advancements, the traditional rule-based approaches and the transformer-based models face several challenges, namely high annotation costs, generalization issues, and hallucinations in the LLMs. The traditional systems require extensive manual annotation, making them resource-intensive and difficult to scale. The transformer-based models demand large amounts of labelled data and may struggle with underrepresented biomedical subdomains, leading to inconsistencies in extraction accuracy. While the LLMs infer relationships beyond explicitly stated facts, they sometimes generate incorrect or misleading information, necessitating additional validation and filtering mechanisms.

There is, therefore, a need in the art to provide an improved system and a method to extract, analyse, and validate biomedical relationships from biomedical literature by overcoming the deficiencies of the prior art(s).

### SUMMARY

The following presents a simplified summary of various aspects of this disclosure in order to provide a basic understanding of such aspects. This summary is not an extensive overview of the disclosure. It is intended to neither identify key or critical elements of the disclosure, nor delineate any scope of the particular implementations of the disclosure or any scope of the claims. Its sole purpose is to present some concepts of the disclosure in a simplified form as a prelude to the more detailed description that is presented later.

In an aspect, the present disclosure relates to a computer-implemented method for extracting and validating clinical relationship triplets from a biomedical literature. The method includes retrieving, by a processor associated with a system, biomedical abstracts from one or more databases associated with the biomedical literature using one or more automated tools. The method includes extracting, by the processor, relationship triplets from the retrieved biomedical abstracts, and validating, by the processor, the extracted relationship triplets through a multi-stage evaluation framework. Further, the method includes facilitating, by the processor, personalized dietary recommendations to one or more users by structuring and outputting the validated relationship triplets.

In one embodiment, the method may include retrieving, by the processor, the biomedical abstracts through web scraping frameworks and Application Programming Interfaces (APIs).

In one embodiment, the retrieved biomedical abstracts may include at least one of an identifier, a title of an article, a summary of the article, a source of publication of the article, a publication type, funding information, and a direct link to the article.

In one embodiment, for extracting, by the processor, the relationship triplets from the retrieved biomedical abstracts, the method may include scraping, by the processor, the retrieved biomedical abstracts, processing one or more texts in the retrieved biomedical abstracts for relationship extraction using a combination of Named Entity Recognition (NER) and relationship extraction models, and a domain-specific transformer-based model, upon scraping the retrieved biomedical abstracts, and generating, by the processor, the relationship triplets using a Large Language Model (LLM) configured for semantic understanding of the one or more texts in the retrieved biomedical abstracts. The relationship extraction model may be at least a Chain-of-Thought (CoT) relationship extraction model.

In one embodiment, prior to extracting the relationship triplets from the retrieved biomedical abstracts, the method may include pre-processing, by the processor, the one or more texts in the retrieved biomedical abstracts by splitting the retrieved biomedical abstracts into one or more individual sentences, and converting all texts in each of the one or more individual sentences to a lowercase for uniformity.

In one embodiment, upon pre-processing the one or more texts in the retrieved biomedical abstracts, the method may include annotating, by the processor, the one or more texts in the retrieved biomedical abstracts using an annotation tool. Upon annotating the one or more texts, the method may include detecting, by the processor, biomedical terms in the retrieved biomedical abstracts by applying NER to the retrieved biomedical abstracts, and annotating, by the processor, positive and negative relationships among the biomedical terms by tagging one or more entities on the annotation tool.

In one embodiment, the method may include post-processing, by the processor, the extracted relationship triplets by identifying, by the processor, names of one or more features from the extracted relationship triplets, where the one or more features may include at least one of an ingredient and properties associated with the ingredient. Further, the method may include standardizing the names of the one or more features using the LLM, eliminating duplicates and contradictory relationships from the extracted relationship triplets, and managing the contradictory relationships by retaining maximum support relationships among the extracted relationship triplets using positive or negative tagging techniques.

In one embodiment, for validating, by the processor, the extracted relationship triplets through the multi-stage evaluation framework, the method may include filtering, by the processor, low-quality triplets from the extracted relationship triplets using a machine-learning classifier trained on expert graded biomedical data. Upon filtering, the method may include applying chain-of-thought reasoning via the LLM to assign a clinically validated grading score to each relationship triplet among the extracted relationship triplets based on one or more parameters, and prioritizing the extracted relationship triplets based on the clinically validated grading score. Upon prioritizing, the method may include verifying factual accuracy of each relationship triplet using a medical question-answering model, where the medical question-answering model cross-references the extracted relationship triplets against an original abstract content to validate the extracted relationship triplets.

In one embodiment, for filtering, by the processor, the low-quality triplets from the extracted relationship triplets, the method may include distinguishing valid relationships and noisy relationships from the extracted relationship triplets based on manual grading of samples randomly selected from a post-processed relationship dataset by experts, and identifying noisy relationships as the low-quality triplets.

In one embodiment, prior to applying the chain-of-thought reasoning, via the LLM, the method may include performing LLM-based grading to identify biased relationships from the extracted relationship triplets, and discarding the biased relationships using an automated sponsor-bias filter.

In one embodiment, for verifying, by the processor, the factual accuracy of each relationship triplet, the method may include providing each relationship triplet among the extracted relationship triplets as contextual input into the LLM model, converting each relationship triplet into a query, passing the query as a prompt into the LLM model, and receiving a logical response indicating whether the relationship of each relationship triplet is factually correct from the LLM model.

In one embodiment, the logical response may be at least one of a positive response or a negative response.

In one embodiment, the method may include confirming, by the processor, the factual accuracy of each relationship triplet as valid and retaining each valid relationship triplet if the logical response is the positive response. Further, the method may include discarding, by the processor, the relationship triplets if the logical response is the negative response.

In one embodiment, the method may include facilitating, by the processor, the personalized dietary recommendations to the one or more users to enable the one or more users to make informed decisions regarding at least diet, supplements, and health management.

In an aspect, the present disclosure relates to a system for extracting and validating clinical relationship triplets from a biomedical literature. The system includes a processor and a memory operatively coupled to the processor. The memory comprises processor-executable instructions, which on execution, cause the processor to retrieve, using an abstract retrieval module, biomedical abstracts from one or more databases associated with the biomedical literature using one or more automated tools. The processor extracts, using a relationship extraction module, relationship triplets from the retrieved biomedical abstracts, and validates, using a multi-layer validation module, the extracted relationship triplets through a multi-stage evaluation framework. Further, the processor facilitates personalized dietary recommendations to one or more users by structuring and outputting the validated relationship triplets.

In one embodiment, the processor may be configured to standardize, deduplicate, and reconcile conflicting triplets among the extracted relationship triplets using a post-processing module.

In one embodiment, to extract the relationship triplets from the retrieved biomedical abstracts, the processor may be configured to scrape the retrieved biomedical abstracts, and processing one or more texts in the retrieved biomedical abstracts for relationship extraction using a combination of NER and relationship extraction models, and a domain-specific transformer-based model, upon scraping the retrieved biomedical abstracts. The relationship extraction model may be at least a Chain-of-Thought (CoT) relationship extraction model.

The processor may be configured to generate the relationship triplets using an LLM configured for semantic understanding of the one or more texts in the retrieved biomedical abstracts.

In one embodiment, to validate the extracted relationship triplets through the multi-stage evaluation framework, the processor is configured to filter low-quality triplets from the extracted relationship triplets using a machine-learning classifier trained on expert graded biomedical data. Upon filtering, the processor is configured to apply chain-of-thought reasoning via an LLM to assign a clinically validated grading score to each relationship triplet among the extracted relationship triplets based on one or more parameters, prioritize the extracted relationship triplets based on the clinically validated grading score, and verify factual accuracy of each relationship triplet using a medical question-answering model. The medical question-answering model may cross-reference the extracted relationship triplets against an original abstract content to validate the extracted relationship triplets.

In one embodiment, to filter the low-quality triplets from the extracted relationship triplets, the processor is configured to distinguish valid relationships and noisy relationships from the extracted relationship triplets based on manual grading of samples randomly selected from a post-processed relationship dataset by experts, and identify noisy relationships as the low-quality triplets.

In one embodiment, prior to applying the chain-of-thought reasoning, via the LLM, the processor is configured to perform LLM-based grading to identify biased relationships from the extracted relationship triplets, and discard the biased relationships using an automated sponsor-bias filter.

In one embodiment, to verify the factual accuracy of each relationship triplet, the processor is configured to provide each relationship triplet among the extracted relationship triplets as contextual input into the LLM model, convert each relationship triplet into a query, pass the query as a prompt into the LLM model, and receive a logical response indicating whether the relationship of each relationship triplet is factually correct from the AI model. The logical response may be at least one of a positive response or a negative response.

In one embodiment, the processor may be configured to confirm the factual accuracy of each relationship triplet as valid and retain each valid relationship triplet if the logical response is the positive response, and discard the relationship triplets if the logical response is the negative response.

In one embodiment, the processor may be configured to validate the extracted relationship triplets through the multi-stage evaluation framework, using at least a CoT-based relationship validation model.

### BRIEF DESCRIPTION OF DRAWINGS

The accompanying drawings, which are incorporated herein, and constitute a part of this disclosure, illustrate exemplary embodiments of the disclosed methods and systems which like reference numerals refer to the same parts throughout the different drawings. Components in the drawings are not necessarily to scale, emphasis instead being placed upon clearly illustrating the principles of the present disclosure. Some drawings may indicate the components using block diagrams and may not represent the internal circuitry of each component. It will be appreciated by those skilled in the art that disclosure of such drawings includes the disclosure of electrical components, electronic components, or circuitry commonly used to implement such components.

FIG. 1 illustrates an example block diagram of a system for extracting and validating clinical relationship triplets from a biomedical literature, in accordance with an embodiment of the present disclosure.

FIG. 2 illustrates an exemplary flow diagram for implementing a method for extracting and validating clinical relationship triplets from a biomedical literature, in accordance with embodiments of the present disclosure.

FIG. 3 illustrates an exemplary flow diagram of a method for relationship extraction using traditional Machine Learning (ML) approach and using Large Language Model (LLM) Approach, in accordance with embodiments of the present disclosure.

FIGS. 4A-4C illustrates exemplary flow diagrams of a method for validation using a classifier, Chain-of-Thought LLM, and factual verification, respectively, in accordance with embodiments of the present disclosure.

FIG. 5 illustrates an exemplary computer system in which or with which embodiments of the present disclosure may be utilized in accordance with embodiments of the present disclosure.

The foregoing shall be more apparent from the following more detailed description of the disclosure.

### DETAILED DESCRIPTION

In the following description, for the purposes of explanation, various specific details are set forth in order to provide a thorough understanding of embodiments of the present disclosure. It will be apparent, however, that embodiments of the present disclosure may be practiced without these specific details. Several features described hereafter can each be used independently of one another or with any combination of other features. An individual feature may not address all of the problems discussed above or might address only some of the problems discussed above. Some of the problems discussed above might not be fully addressed by any of the features described herein.

The ensuing description provides exemplary embodiments only and is not intended to limit the scope, applicability, or configuration of the disclosure. Rather, the ensuing description of the exemplary embodiments will provide those skilled in the art with an enabling description for implementing an exemplary embodiment. It should be understood that various changes may be made in the function and arrangement of elements without departing from the spirit and scope of the disclosure as set forth.

Specific details are given in the following description to provide a thorough understanding of the embodiments. However, it will be understood by one of ordinary skill in the art that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail to avoid obscuring the embodiments.

Also, it is noted that individual embodiments may be described as a process that is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed but could have additional steps not included in a figure. A process may correspond to a method, a function, a procedure, a subroutine, a subprogram, etc. When a process corresponds to a function, its termination can correspond to a return of the function to the calling function or the main function.

The word “exemplary” and/or “demonstrative” is used herein to mean serving as an example, instance, or illustration. For the avoidance of doubt, the subject matter disclosed herein is not limited by such examples. In addition, any aspect or design described herein as “exemplary” and/or “demonstrative” is not necessarily to be construed as preferred or advantageous over other aspects or designs, nor is it meant to preclude equivalent exemplary structures and techniques known to those of ordinary skill in the art. Furthermore, to the extent that the terms “includes,” “has,” “contains,” and other similar words are used in either the detailed description or the claims, such terms are intended to be inclusive in a manner similar to the term “comprising” as an open transition word without precluding any additional or other elements.

Reference throughout this specification to “one embodiment” or “an embodiment” or “an instance” or “one instance” means that a particular feature, structure, or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terminology used herein is for the purpose of describing particular embodiments only and is not intended to be limiting of the disclosure. As used herein, the singular forms “a”, “an”, and “the” are intended to include the plural forms as well, unless the context indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Existing technologies, such as rule-based systems and classical machine learning methods, require extensive manual annotation and feature engineering, limiting scalability and adaptability across diverse biomedical subdomains. Although transformer-based models have improved extraction accuracy by learning contextual representations from large datasets, they are highly data-intensive. They may struggle to generalize in underrepresented contexts. Moreover, Large Language Models (LLMs) in zero- or few-shot settings may sometimes generate inferred relationships not explicitly present in a source text, resulting in hallucinations or factual inconsistency. These challenges, high annotation costs, and inconsistent extraction reliability hinder dependable knowledge discovery. Therefore, there is, a need for an improved system and method to address several critical challenges in biomedical relationship extraction, particularly under demanding real-world conditions.

The present disclosure provides a system and a method for extracting and validating clinical relationship triplets from a biomedical literature. The system introduces a comprehensive, multi-stage pipeline that seamlessly integrates traditional machine learning techniques with cutting-edge LLMs to improve an accuracy and reliability of biomedical relationship extraction. The system and the method automatically retrieve biomedical abstracts from one or more databases, for example, but not limited to, PubMed using automated tools such as, for example, without limitation, Application Programming Interface (API). Once retrieved, the biomedical abstracts undergo relationship extraction through a combination of Named Entity Recognition (NER) and relationship extraction models, leveraging transformer-based models alongside a prompt-engineered LLM to enhance contextual understanding. To ensure the extracted relationships are valid and clinically meaningful, the system employs a three-stage validation framework as explained below:
i. Noise Filtering: A machine-learning classifier, for example, a random forest classifier, trained on thousands of expert-annotated biomedical relationships, removes inaccurate or low-quality extractions.
ii. Clinical grading: The LLM applies chain-of-thought (CoT) reasoning, assigning a clinically validated grade score to the biomedical relationships based on study quality and strength of evidence.
iii. Factual verification: A domain-specific medical question-answering model cross-checks the extracted relationships against an original abstract text to ensure factual consistency.

Therefore, the proposed system and the method effectively identify and validate both positive and negative relationships between food ingredients, diseases, biological processes, and health-related properties. By providing high-quality, clinically validated insights, the system empowers consumers, healthcare professionals, and researchers to make more informed decisions about diet, supplements, and general health management. Furthermore, the proposed system and the method enable personalized dietary recommendations by extracting scientifically supported relationships between food components and their effects on health conditions.

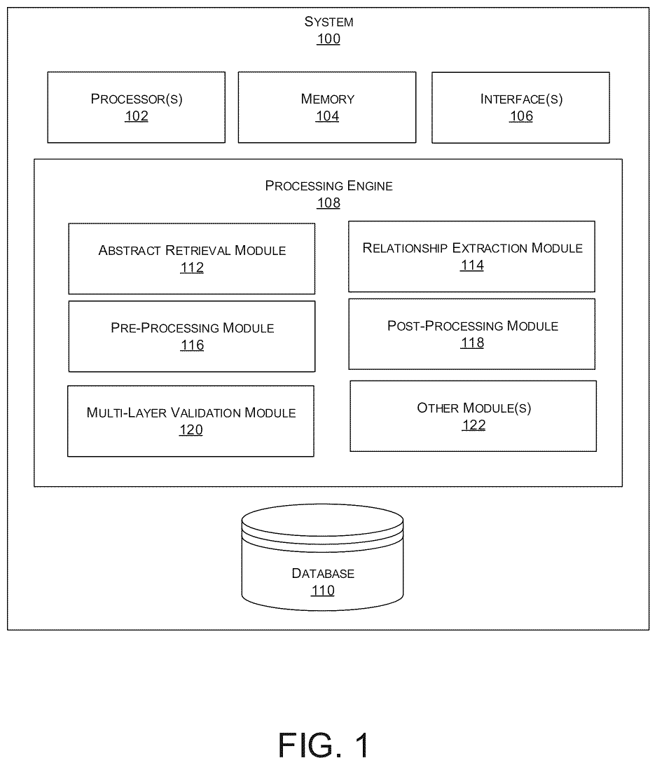

Various embodiments of the present disclosure will be explained in detail with reference to FIGS. 1-5.

FIG. 1 illustrates an exemplary block diagram of a system 100 for extracting and validating clinical relationship triplets from a biomedical literature, in accordance with an embodiment of the present disclosure.

The system 100 combines classical machine learning techniques with modern LLMs to identify relationships between food ingredients and their respective properties, diseases, or processes.

With reference to FIG. 1, in an embodiment, the system 100 may include a processor 102. The processor 102 may be implemented as one or more microprocessors, microcomputers, microcontrollers, digital signal processors, central processing units, logic circuitries, and/or any devices that manipulate data based on operational instructions. Among other capabilities, the processor 102 may be configured to fetch and execute computer-readable instructions stored in a memory 104 of the system 100. The memory 104 may store one or more computer-readable instructions or routines, which may be fetched and executed to create or share the data units over a network service. The memory 104 may include any non-transitory storage device including, for example, a volatile memory such as a Random-Access Memory (RAM), or a non-volatile memory such as an Erasable Programmable Read-Only Memory (EPROM), a flash memory, and the like.

In an embodiment, the system 100 may also include an interface(s) 106. The interface(s) 106 may include a variety of interfaces, for example, a variety of interfaces, for example, interfaces for data input and output devices, referred to as I/O devices, storage devices, and the like. The interface(s) 106 may facilitate communication of the system 100 with various devices coupled to it. The interface(s) 106 may also provide a communication pathway for one or more components of the system 100. Examples of such components include, but are not limited to, processing engine(s) 108 and a database 110.

In an embodiment, the processing engine(s) 108 may be implemented as a combination of hardware and programming (for example, programmable instructions) to implement one or more functionalities of the processing engine(s) 108. In examples, described herein, such combinations of hardware and programming may be implemented in several different ways. For example, the programming for the processing engine(s) 108 may be processor-executable instructions stored on a non-transitory machine-readable storage medium and the hardware for the processor 102 may include a processing resource, to execute such instructions. In the present examples, the machine-readable storage medium may store instructions that, when executed by the processing resource, implement the processing engine(s) 108. In such examples, the system 100 may comprise the machine-readable storage medium storing the instructions and the processing resource to execute the instructions, or the machine-readable storage medium may be separate but accessible to the system 100 and the processing resource. In other examples, the processing engine(s) 108 may be implemented by an electronic circuitry.

In an embodiment, the database 110 may comprise data that may be either stored or generated as a result of functionalities implemented by any of the components of the processors 102 or the processing engine(s) 108 or the system 100.

In an exemplary embodiment, the processing engine(s) 108 may include one or more modules selected from any of an abstract retrieval module 112, a relationship extraction module 114, a pre-processing module 116, a post-processing module 118, a multi-layer validation module 120, and other units/modules 122. The other units/engines 122 may include, but are not limited to, a monitoring module, a determination module, and the like.

In an embodiment, the processor 102 may, via the abstract retrieval module 112, retrieve biomedical abstracts from one or more databases associated with the biomedical literature using one or more automated tools. In an embodiment, the processor 102 may, via the relationship extraction module 114, extract relationship triplets from the retrieved biomedical abstracts. The relationship triplet may be a structured way to represent relationships between biomedical entities found in clinical or biomedical literature. The triplets may consist of three components, namely, a first biomedical entity (for e.g., without limitation, a food ingredient, food product, drug, or ethnicity), a relation-a type of interaction or association between the two entities (e.g., “reduces risk of,” “causes,” “treats,” “associated with”), and a second biomedical entity (for e.g., without limitation, a disease, symptom, a biological process, or food-ingredient properties).

In an embodiment, the processor 102 may, via the pre-processing module 116, pre-process one or more texts in the retrieved biomedical abstracts by splitting the retrieved biomedical abstracts into one or more individual sentences, and converting all texts in each of the one or more individual sentences to a lowercase for uniformity.

In an embodiment, the processor 102 may, via the post-processing module 118, standardize, deduplicate, and reconcile conflicting triplets among the extracted relationship triplets. In an embodiment, the processor 102 may, via the multi-layer validation module 120, validate the extracted relationship triplets through a multi-stage evaluation framework. The processor 102 may, via the multi-layer validation module 120, validate the extracted relationship triplets using at least a CoT-based relationship validation model. Further, the processor 102 may facilitate personalized dietary recommendations to one or more users by structuring and outputting the validated relationship triplets. The one or more users may be, for example, consumers, healthcare professionals, and researchers. The one or more users may make more informed decisions about diet, supplements, and general health management using the personalized dietary recommendations.

Although FIG. 1 shows exemplary components of the system 100, in other embodiments, the system 100 may include fewer components, different components, differently arranged components, or additional functional components than depicted in FIG. 1. Additionally, or alternatively, one or more components of the system 100 may perform functions described as being performed by one or more other components of the system 100.

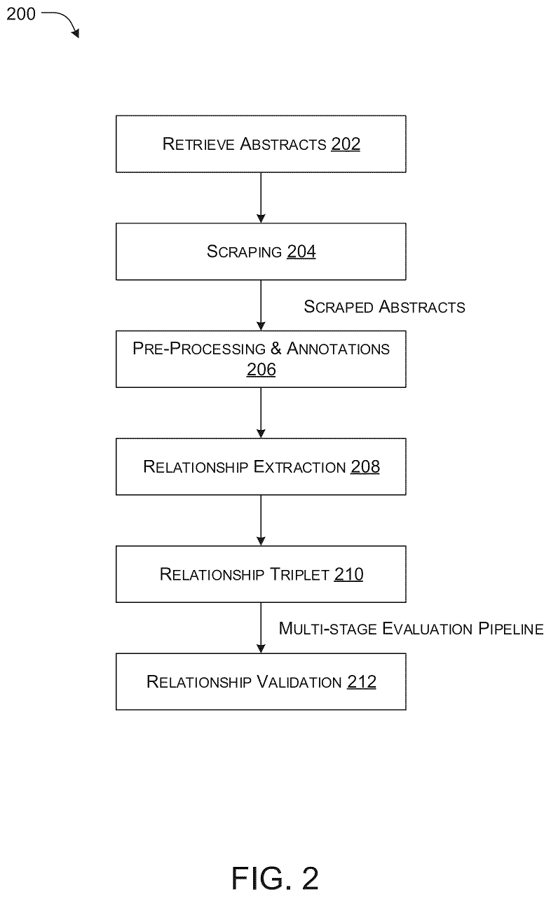

FIG. 2 illustrates an exemplary flow diagram for implementing a method 200 for extracting and validating clinical relationship triplets from the biomedical literature, in accordance with embodiments of the present disclosure.

With reference to FIG. 2, the method 200 may include one or more steps. The one or more steps may be performed by the modules of the system 100 as illustrated in FIG. 1.

At step 202, the method 200 may include retrieving biomedical abstracts from one or more databases associated with the biomedical literature using one or more automated tools. The biomedical abstracts may be retrieved through web scraping frameworks and APIs. The retrieved biomedical abstracts may include, but not limited to, an identifier, a title of an article, a summary of the article, a source of publication of the article, a publication type, funding information, and a direct link to the article.

At step 204, the method 200 may include scraping the retrieved biomedical abstracts. Scraping the retrieved biomedical abstracts may refer to an automated extraction of structured text data including one or more texts from the databases. The scraping may allow the users to efficiently collect relevant studies for further processing, such as for relationship extraction, text mining, etc.

At step 206, the method 200 may include pre-processing the one or more texts in the retrieved biomedical abstracts. The one or more texts may be pre-processed by splitting the retrieved biomedical abstracts into one or more individual sentences, and converting all texts in each of the one or more individual sentences to a lowercase for uniformity. Further, the method 200 may include annotating the one or more texts in the retrieved biomedical abstracts using an annotation tool. Upon annotating the one or more texts, the method 200 may include detecting biomedical terms in the retrieved biomedical abstracts by applying NER to the retrieved biomedical abstracts, and annotating positive and negative relationships among the biomedical terms by tagging one or more entities on the annotation tool. The one or more entities may include, without limitation, ingredients (food ingredients (e.g., turmeric, ginger, fish oil, etc.,), diseases (e.g., cancer, diabetes, etc), natural body processes (e.g., blood pressure, heart health, etc.,), or properties (e.g., anti-inflammatory, antioxidant, etc.,).

At step 208, the method 200 may include extracting relationship triplets from the retrieved biomedical abstracts. The relationship triplets may be extracted by processing the one or more texts in the retrieved biomedical abstracts using a combination of NER and relationship extraction models, and a domain-specific transformer-based model, and generating the relationship triplets using the LLM configured for semantic understanding of the one or more texts in the retrieved biomedical abstracts. The relationship extraction model is at least a Chain-of-Thought (CoT) relationship extraction model.

At step 210, the method 200 may include post-processing the extracted relationship triplets. The extracted relationship triplets may be post-processed by identifying names of the one or more entities from the extracted relationship triplets, and standardizing the names of the one or more entities using the LLM. Upon standardizing the names, the method 200 may include eliminating duplicates and contradictory relationships from the extracted relationship triplets, and managing the contradictory relationships by retaining maximum support relationships among the extracted relationship triplets using positive or negative tagging techniques.

At step 212, the method 200 may include validating the extracted relationship triplets through a multi-stage evaluation framework.

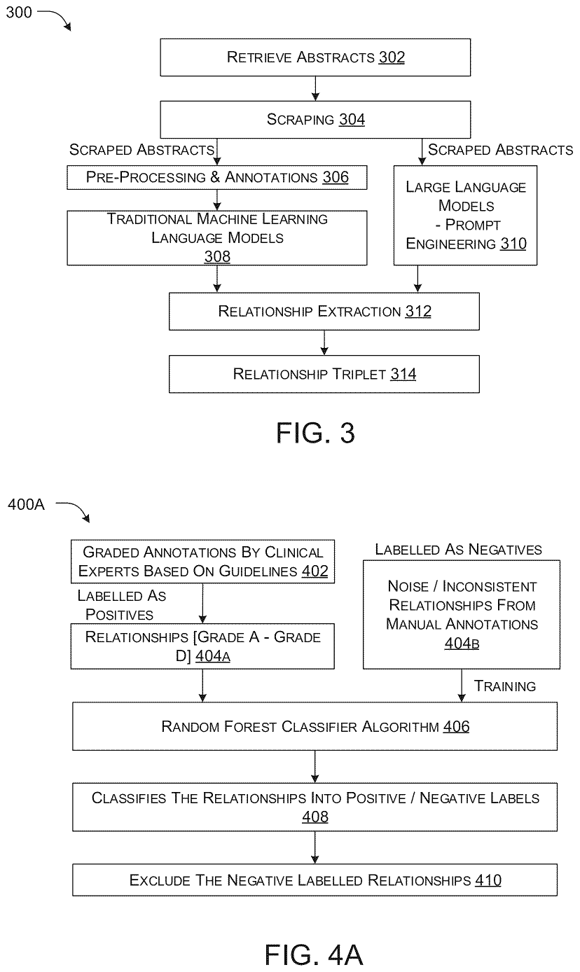

FIG. 3 illustrates an exemplary flow diagram of a method 300 for relationship extraction using traditional Machine Learning (ML) approach and using LLM approach, in accordance with embodiments of the present disclosure.

With reference to FIG. 3, the method 300 may include one or more steps. The one or more steps may be performed by using the relationship extraction module 114 of the system 100, as illustrated in FIG. 1.

At step 302, the method 300 may include retrieving biomedical abstracts from the databases associated with the biomedical literature using the automated tools. The method 300 may apply filters to retrieve only clinically relevant abstracts such as clinical trials, randomized controlled trials (RCTs), double-blinded studies, and systematic reviews.

At step 304, the method 300 may include scraping the retrieved biomedical abstracts through the web scraping frameworks and the APIs.

At step 306, the method 300 may include performing pre-processing and annotation using any of traditional ML approaches. Before extracting relationships, the raw abstract text may be pre-processed through sentence tokenization where the abstracts may be split into individual sentences, and lowercasing where the texts are converted to lowercase for uniformity. After pre-processing, a customized end-to-end NER pipeline may be applied to detect biomedical terms after manual annotations using the annotation tool. Entities may be categorized into two main types, namely, ingredient (E1)→food ingredients (e.g., turmeric, ginger, fish oil), and disease/process/property (E2)→diseases (e.g., cancer, diabetes) or natural body processes (e.g., blood pressure, heart health) or properties (e.g., anti-inflammatory, antioxidant). Further, the method 300 may include annotating relationships after tagging the entities on the UBIAI annotation tool. Both positive and negative relationships may be tagged for the entities E1 and E2. For example, Relationship (r)→describes how entity E1 interacts with entity E2 (e.g., “reduces”, “enhances”, “inhibits”, “causes”).

At step 308, the method 300 may include partitioning manually annotated dataset into training, development, and testing subsets. Pre-trained weights for traditional ML language models may be sourced from a library. These transformer models may be jointly fine-tuned on the annotated training dataset to perform NER and relationship extraction tasks. After fine-tuning, the best-performing model may be evaluated using the test set, and the finalized models may be subsequently applied to unseen abstracts to extract relationships.

The entire training process may be conducted using an open-source Natural Language Processing (NLP) library, with training iterations increased as additional annotations are incorporated. However, the NLP library has certain limitations, such as catastrophic forgetting, which occurs during retraining of a NER component, preventing an ability to freeze specific labels within the component. Due to these limitations, the LLM-based approach may be recommended for the next phase of relationship extraction.

At step 310, the method 300 may include pre-training LLMs on vast and diverse data, enabling the LLM to generalize effectively to new tasks with minimal task-specific fine-tuning. The LLMs may perform tasks without explicit supervised training on task-specific data. The LLM's pre-training on extensive datasets may enable them to understand and follow instructions via prompts, reducing a need for a large, annotated dataset and extensive retraining. A single example with a published abstract and relationship triplets may be provided, and the LLM may learn from the provided example. The performance of the LLMs in a zero-shot setting heavily relies on carefully crafted prompts. System prompts may be provided to instruct the model on identifying and categorizing entities and their relationships directly from text. For example: System Prompt: Carefully identify and categorize specific entities and their direct relationships from the abstract {abstract}.

The LLMs may require a few data to no task-specific training. Unlike supervised transformer models that demand extensive annotated data and iterative fine-tuning, the zero-shot capability of the LLMs may allow immediate deployment with minimal configuration, saving both time and computational resources. Due to diverse pre-training of the LLMs, the LLMs can generalize better to unseen data and handle text variations that might require additional annotations or model retraining in conventional approaches. Further, the prompt-engineered approach may streamline the extraction process. There is no need to partition datasets or manage issues like catastrophic forgetting, as observed with frameworks like in the NLP library in the previous method.

At step 312, the method 300 may include performing relationship extraction. The relationship extraction may be performed by identifying names of the entities from the retrieved biomedical abstracts using the domain-specific transformer-based model. The names of the entities may be standardized using the LLMs. Upon standardizing the names, the method 300 may include eliminating duplicates and contradictory relationships from the retrieved biomedical abstracts, and managing the contradictory relationships by retaining maximum support relationships among the retrieved biomedical abstracts using positive or negative tagging techniques.

At step 314, the method 300 may include processing the texts in the retrieved biomedical abstracts using a combination of NER and relationship extraction models along with the domain-specific transformer-based model, upon scraping the retrieved biomedical abstracts. The method 300 may include generating the relationship triplets using the LLM configured for semantic understanding of the texts in the retrieved biomedical abstracts, and extracting the relationship triplets from the retrieved biomedical abstracts.

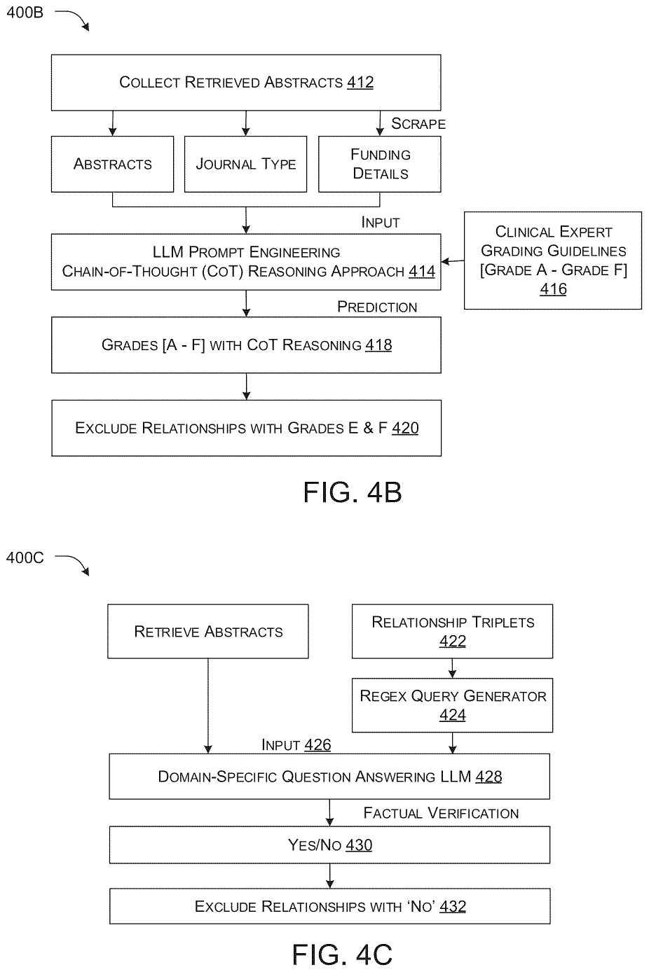

FIGS. 4A-4C illustrates exemplary flow diagrams of methods 400A-400C for validation using a classifier, Chain-of-Thought LLM, and factual verification, respectively, in accordance with embodiments of the present disclosure.

With reference to FIGS. 4A-4C, the validation methods 400A-400C may include one or more steps. The one or more steps may be performed by the multi-layer validation module 120 of the system, as illustrated in FIG. 1.

With reference to FIG. 4A, once the relationship triplets are extracted, the extracted relationship triplets may be validated through the multi-stage evaluation framework. At 402, the validation method 400A may include filtering low-quality triplets from the extracted relationship triplets using a machine-learning classifier trained on expert graded biomedical data. Filtering may be performed by distinguishing valid relationships and noisy relationships from the extracted relationship triplets, and identifying the noisy relationships as the low-quality triplets. To facilitate filtering, clinical experts has manually graded a few randomly selected samples from the post-processed relationship dataset based on the following grading criteria:
i. Grade A: The paper abstract is a Systematic Review.
ii. Grade B: The paper abstract is an RCT conducted as a Multi-Centre study.
iii. Grade C: The paper abstract is an RCT from a Single Centre with over 100 participants.
iv. Grade D: The paper abstract is an RCT from a Single Centre with fewer than 100 participants.
v. Grade E: The paper abstract is an Observational Study.
vi. Grade F (Unusable): The paper abstract is deemed unusable if any of the following conditions are met:
   Incorrect ingredient: The paper does not investigate the ingredients listed.
   Incorrect disease: The paper does not investigate the disease listed.
   Incorrect relationship: The reported relationship is inaccurate (e.g., the abstract suggests a positive association when there is none or a negative association exists).
   Bias sponsor: The study is biased due to sponsorship (e.g., the beef industry sponsoring a study demonstrating beef lowers cholesterol).
   Scoping or literature review: The paper is a scoping or literature review.
   Not statistically significant: The study results are not statistically significant (e.g., confidence interval crossing 1).

At step 404a, the validation method 400A may include selecting and labelling the relationships graded A through D as Positive. At step 404b, in contrast, the validation method 400A may include labelling the relationships identified as Grade E, F, and inconsistent or incorrect relationships during the initial post-processing (and consequently discarded) as Negative. The inconsistent or incorrect relationships may be removed and tracked separately before grading the relationships. At step 406, the validation method 400A may include evaluating multiple classifiers on the expert-graded dataset, and determining performance of the random forest classifier as superior. At step 408, the validation method 400A may include using the random forest classifier to reliably identify and classify the relationships positive/negative labels. At step 410, the validation method 400A may include discarding negative relationships from the post-processed dataset during testing.

With reference to FIG. 4B, upon filtering, the validation method 400B may include applying chain-of-thought reasoning via the LLM to assign a clinically validated grading score to each relationship triplet among the extracted relationship triplets based on one or more parameters, and prioritizing the extracted relationship triplets based on the clinically validated grading score. In an embodiment, prior to applying the chain-of-thought reasoning, via the LLM, the validation method 400B may include performing LLM-based grading to identify biased relationships from the extracted relationship triplets, and discarding the biased relationships using an automated sponsor-bias filter. Further, the validation method 400B may include verifying factual accuracy of each relationship triplet using a medical question-answering model. The medical question-answering model may cross-reference the extracted relationship triplets against an original abstract content to validate the extracted relationship triplets.

The validation method 400B may include utilizing the LLM configured with a chain-of-thought reasoning framework to emulate clinical expert evaluations of relationships. The validation method 400B may be structured as follows. At step 412, the method 400B may include collecting the retrieved biomedical abstracts including, but not limited to, the identifier, the title of the article, the summary of the article, the source of publication of the article, the publication type, funding information, and the direct link to the article.

At step 414, the method 400B may include performing prompt configuration using the chain-of-thought reasoning framework, which includes:
i. System Prompt: The LLM may be assigned a role of a clinical expert relationship evaluator.
ii. General Prompt: The grading criteria (as defined in the previous section) may be provided.
iii. Input Data: Each retrieved biomedical abstract and the relationship triplets may be passed into the prompt alongside these instructions.

At step 416, while the LLM generates grades for relationships, there exists a possibility of hallucination or reliance on probabilistic predictions. The method 400B may include articulating a step-by-step reasoning process for each grade prediction to mitigate the possibility of hallucination or reliance on the probabilistic predictions using the LLM. This approach may ensure that each grade is supported by a clear, logical explanation aligned with the established grading criteria.

At step 418, the method 400B may include implementing a filtering step after the LLM assigns grades with corresponding justifications. At step 420, the method 400B may include retaining only relationships with grades A through D, while the relationships with other grades may be discarded. Therefore, the method 400B may enhance the reliability of the grading process by combining expert-level guidance with explicit reasoning, thereby reducing a risk of spurious or probabilistically driven predictions.

With reference to FIG. 4C, the validation method 400C may employ a domain-specific question-answering framework using the LLM model, which may be fine-tuned for medical tasks and built upon the LLaMA architecture. The factual accuracy of each relationship triplet may be verified by following steps.

At step 422, the validation method 400C may include providing each relationship triplet as contextual input into the LLM model. At step 424, the validation method 400C may include applying Regex-based transformations to generate queries by converting each relationship triplet into a query. At step 426, the validation method 400C may include passing the query as a prompt into the LLM model. At step 428, the LLM model may receive the prompt as an input. At step 430, the LLM model may send a logical response indicating whether the relationship of each relationship triplet is factually correct or not to the system 100. The logical response may be a positive response (“Yes”) or a negative response (“No”). The logical response may be, for example, but not limited to, a binary response, a Boolean value, or a response in a Natural Language form. At step 432, if the logical response is the positive response, the validation method 400C may include confirming the factual accuracy of each relationship triplet as valid and retaining each valid relationship triplet. If the logical response is the negative response, the validation method 400C may include discarding the relationship triplets.

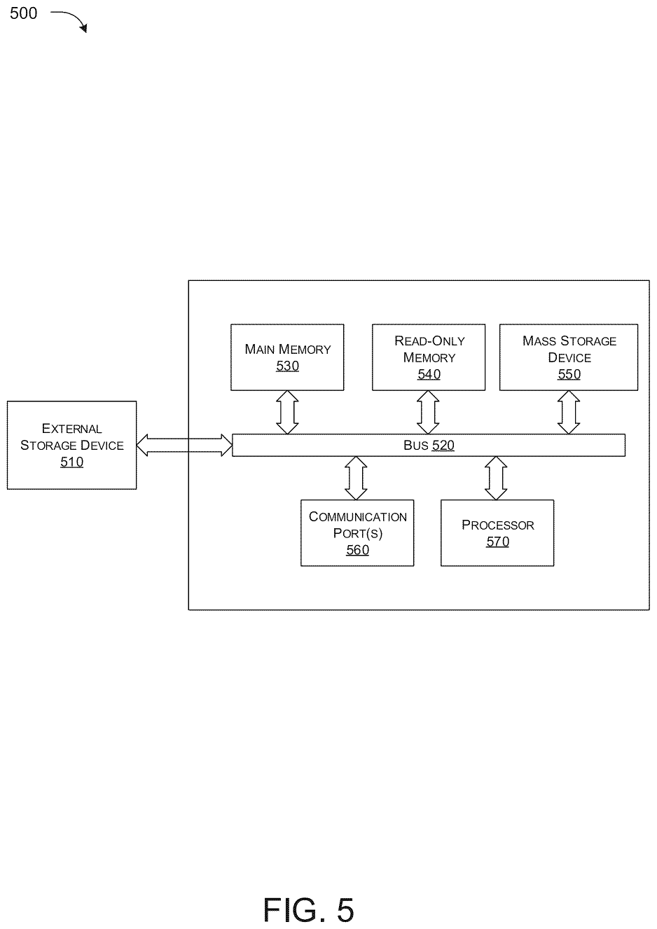

FIG. 5 illustrates an exemplary computer system 500 in which or with which embodiments of the present disclosure may be utilized in accordance with embodiments of the present disclosure.

As shown in FIG. 5, the computer system 500 may include an external storage device 510, a bus 520, a main memory 530, a read-only memory 540, a mass storage device 550, a communication port(s) 560, and a processor 570. A person skilled in the art will appreciate that the computer system 500 may include more than one processor 570 and communication ports 560. The processor 570 may include various modules associated with embodiments of the present disclosure. The processor 570 may be, for example, but not limited to, a Graphics Processing Unit (GPU). The communication port(s) 560 may be any of an RS-232 port for use with a modem-based dialup connection, a 10/100 Ethernet port, a Gigabit or 10 Gigabit port using copper or fiber, a serial port, a parallel port, or other existing or future ports. The communication ports(s) 560 may be chosen depending on a network, such as a Local Area Network (LAN), Wide Area Network (WAN), or any network to which the computer system 500 connects.

In an embodiment, the main memory 530 may be a Random-Access Memory (RAM), or any other dynamic storage device commonly known in the art. The read-only memory 540 may be any static storage device(s) e.g., but not limited to, a Programmable Read Only Memory (PROM) chip for storing static information e.g., start-up or basic input/output system (BIOS) instructions for the processor 570. The mass storage device 550 may be any current or future mass storage solution, which can be used to store information and/or instructions. Exemplary mass storage solutions include, but are not limited to, Parallel Advanced Technology Attachment (PATA) or Serial Advanced Technology Attachment (SATA) hard disk drives or solid-state drives (internal or external, e.g., having Universal Serial Bus (USB) and/or Firewire interfaces).

In an embodiment, the bus 520 may communicatively couple the processor(s) 570 with the other memory, storage, and communication blocks. The bus 520 may be, e.g., a Peripheral Component Interconnect (PCI)/PCI Extended (PCI-X) bus, Small Computer System Interface (SCSI), USB, or the like, for connecting expansion cards, drives, and other subsystems as well as other buses, such a front side bus (FSB), which connects the processor 570 to the computer system 500.

In another embodiment, operator and administrative interfaces, e.g., a display, keyboard, and cursor control device may also be coupled to the bus 520 to support direct operator interaction with the computer system 500. Other operator and administrative interfaces can be provided through network connections connected through the communication port(s) 560. Components described above are meant only to exemplify various possibilities. In no way should the aforementioned exemplary computer system 500 limit the scope of the present disclosure.

While considerable emphasis has been placed herein on the preferred embodiments, it will be appreciated that many embodiments can be made and that many changes can be made in the preferred embodiments without departing from the principles of the disclosure. These and other changes in the preferred embodiments of the disclosure will be apparent to those skilled in the art from the disclosure herein, whereby it is to be distinctly understood that the foregoing descriptive matter is to be implemented merely as illustrative of the disclosure and not as a limitation.

We claim:

1. A computer-implemented method for extracting and validating clinical relationship triplets from a biomedical literature, the method comprising:
retrieving, by a processor associated with a system, biomedical abstracts from one or more databases associated with the biomedical literature using one or more automated tools;
extracting, by the processor, relationship triplets from the retrieved biomedical abstracts;
validating, by the processor, the extracted relationship triplets through a multi-stage evaluation framework; and
facilitating, by the processor, personalized dietary recommendations to one or more users by structuring and outputting the validated relationship triplets.

2. The method of claim 1, wherein retrieving, by the processor, the biomedical abstracts through web scraping frameworks and Application Programming Interfaces (APIs).

3. The method of claim 1, wherein the retrieved biomedical abstracts comprise at least one of: an identifier, a title of an article, a summary of the article, a source of publication of the article, a publication type, funding information, and a direct link to the article.

4. The method of claim 1, wherein for extracting, by the processor, the relationship triplets from the retrieved biomedical abstracts, the method comprises:
scraping, by the processor, the retrieved biomedical abstracts;
processing, by the processor, one or more texts in the retrieved biomedical abstracts for relationship extraction using a combination of Named Entity Recognition (NER) and relationship extraction models, and a domain-specific transformer-based model, upon scraping the retrieved biomedical abstracts, wherein the relationship extraction model is at least a Chain-of-Thought (CoT) Relationship Extraction model; and
generating, by the processor, the relationship triplets using a Large Language Model (LLM) configured for semantic understanding of the one or more texts in the retrieved biomedical abstracts.

5. The method of claim 4, wherein prior to extracting the relationship triplets from the retrieved biomedical abstracts, the method comprises:
pre-processing, by the processor, the one or more texts in the retrieved biomedical abstracts by splitting the retrieved biomedical abstracts into one or more individual sentences, and converting all texts in each of the one or more individual sentences to a lowercase for uniformity.

6. The method of claim 5, wherein upon pre-processing the one or more texts in the retrieved biomedical abstracts, the method comprises:
annotating, by the processor, the one or more texts in the retrieved biomedical abstracts using an annotation tool;
upon annotating the one or more texts, detecting, by the processor, biomedical terms in the retrieved biomedical abstracts by applying NER to the retrieved biomedical abstracts; and
annotating, by the processor, positive and negative relationships among the biomedical terms by tagging one or more entities on the annotation tool.

7. The method of claim 4, wherein the method comprises post-processing, by the processor, the extracted relationship triplets by:
identifying, by the processor, names of one or more entities from the extracted relationship triplets;
standardizing, by the processor, the names of the one or more entities using the LLM;
upon standardizing the names, eliminating, by the processor, duplicates and contradictory relationships from the extracted relationship triplets; and
managing, by the processor, the contradictory relationships by retaining maximum support relationships among the extracted relationship triplets using positive or negative tagging techniques.

8. The method of claim 1, wherein for validating, by the processor, the extracted relationship triplets through the multi-stage evaluation framework, the method comprises:
filtering, by the processor, low-quality triplets from the extracted relationship triplets using a machine-learning classifier trained on expert graded biomedical data;
upon filtering, applying, by the processor, chain-of-thought reasoning via an LLM to assign a clinically validated grading score to each relationship triplet among the extracted relationship triplets based on one or more parameters;
prioritizing, by the processor, the extracted relationship triplets based on the clinically validated grading score; and
upon prioritizing, verifying, by the processor, factual accuracy of each relationship triplet using a medical question-answering model, wherein the medical question-answering model cross-references the extracted relationship triplets against an original abstract content to validate the extracted relationship triplets.

9. The method of claim 8, wherein for filtering, by the processor, the low-quality triplets from the extracted relationship triplets, the method comprises:
distinguishing, by the processor, valid relationships and noisy relationships from the extracted relationship triplets based on manual grading of samples randomly selected from a post-processed relationship dataset by experts; and
identifying, by the processor, the noisy relationships as the low-quality triplets.

10. The method of claim 8, wherein prior to applying the chain-of-thought reasoning, via the LLM, the method comprises:
performing, by the processor, LLM-based grading to identify biased relationships from the extracted relationship triplets; and
discarding, by the processor, the biased relationships using an automated sponsor-bias filter.

11. The method of claim 1, wherein for verifying, by the processor, the factual accuracy of each relationship triplet, the method comprises:
providing, by the processor, each relationship triplet among the extracted relationship triplets as contextual input into an LLM model;
converting, by the processor, each relationship triplet into a query, and passing the query as a prompt into the LLM model; and
receiving, by the processor, a logical response indicating whether the relationship of each relationship triplet is factually correct from the LLM model.

12. The method of claim 11, wherein the logical response is at least one of: a positive response or a negative response.

13. The method of claim 11, wherein the method comprises:
confirming, by the processor, the factual accuracy of each relationship triplet as valid and retaining each valid relationship triplet if the logical response is the positive response; and
discarding, by the processor, the relationship triplets if the logical response is the negative response.

14. The method of claim 1, wherein facilitating, by the processor, the personalized dietary recommendations to the one or more users to enable the one or more users to make informed decisions regarding at least diet, supplements, and health management.

15. A system for extracting and validating clinical relationship triplets from a biomedical literature, the system comprising:
a processor; and
a memory operatively coupled to the processor, wherein the memory comprises processor-executable instructions, which on execution, cause the processor to:
retrieve, using an abstract retrieval module, biomedical abstracts from one or more databases associated with the biomedical literature using one or more automated tools;
extract, using a relationship extraction module, relationship triplets from the retrieved biomedical abstracts;
validate, using a multi-layer validation module, the extracted relationship triplets through a multi-stage evaluation framework; and
facilitate personalized dietary recommendations to one or more users by structuring and outputting the validated relationship triplets.

16. The system of claim 15, wherein the processor is configured to standardize, deduplicate, and reconcile conflicting triplets among the extracted relationship triplets using a post-processing module.

17. The system of claim 15, wherein to extract the relationship triplets from the retrieved biomedical abstracts, the processor is configured to:
scrape the retrieved biomedical abstracts;
process one or more texts in the retrieved biomedical abstracts for relationship extraction using a combination of Named Entity Recognition (NER) and relationship extraction models, and a domain-specific transformer-based model, upon scraping the retrieved biomedical abstracts, wherein the relationship extraction model is at least a Chain-of-Thought (CoT) relationship extraction model; and
generate the relationship triplets using a Large Language Model (LLM) configured for semantic understanding of the one or more texts in the retrieved biomedical abstracts.

18. The system of claim 15, wherein to validate the extracted relationship triplets through the multi-stage evaluation framework, the processor is configured to:
filter low-quality triplets from the extracted relationship triplets using a machine-learning classifier trained on expert graded biomedical data;
upon filtering, apply chain-of-thought reasoning via an LLM to assign a clinically validated grading score to each relationship triplet among the extracted relationship triplets based on one or more parameters;
prioritize the extracted relationship triplets based on the clinically validated grading score; and
upon prioritizing, verify factual accuracy of each relationship triplet using a medical question-answering model, wherein the medical question-answering model cross-references the extracted relationship triplets against an original abstract content to validate the extracted relationship triplets.

19. The system of claim 18, wherein to filter the low-quality triplets from the extracted relationship triplets, the processor is configured to:
distinguish valid relationships and noisy relationships from the extracted relationship triplets based on manual grading of samples randomly selected from a post-processed relationship dataset by experts; and
identify noisy relationships as the low-quality triplets.

20. The system of claim 18, wherein prior to applying the chain-of-thought reasoning, via the LLM, the processor is configured to:
perform LLM-based grading to identify biased relationships from the extracted relationship triplets; and
discard the biased relationships using an automated sponsor-bias filter.

21. The system of claim 15, wherein to verify the factual accuracy of each relationship triplet, the processor is configured to:
provide each relationship triplet among the extracted relationship triplets as contextual input into an LLM model;
convert each relationship triplet into a query, and pass the query as a prompt into the LLM model; and
receive a logical response indicating whether the relationship of each relationship triplet is factually correct from the LLM model, wherein the logical response is at least one of: a positive response or a negative response.

22. The system of claim 18, wherein the processor is configured to:
confirm the factual accuracy of each relationship triplet as valid and retain each valid relationship triplet if the logical response is the positive response; and
discard the relationship triplets if the logical response is the negative response.

23. The system of claim 18, wherein the processor is configured to validate the extracted relationship triplets through the multi-stage evaluation framework, using at least a CoT-based relationship validation model.
